(19) United States
(12) Patent Application Publication
(10) Pub. No.: US 2022/0118197 A1
(43) Pub. Date: Apr. 21, 2022

(54) Mask Enhancing Insert

(71) Applicant: Mary Anderson Chatham Groton, Atlanta, GA (US)

(72) Inventor: Mary Anderson Chatham Groton, Atlanta, GA (US)

(21) Appl. No.: 17/506,417

(22) Filed: Oct. 20, 2021

Related U.S. Application Data
(60) Provisional application No. 63/094,689, filed on Oct. 21, 2020.

(51) Int. Cl. A61M 15/00; A61M 21/02; A61M 21/00

(52) U.S. Cl. CPC A61M 15/0001; A61M 2021/0016; A61M 21/02

(57) ABSTRACT

Embodiments of the disclosure include a removable face mask enhancing insert comprising an absorbent material; an adhesive applied to a first side of the absorbent material; and a solution applied to at least a second side of the absorbent material, wherein the solution is configured to evaporate from the absorbent material.

### CROSS REFERENCE TO RELATED APPLICATION

This application claims the benefit of U.S. Provisional Application No. 63/094,689, filed Oct. 21, 2020, and entitled “MASK ENHANCING INSERT,” which is hereby incorporated by reference in its entirety.

### FIELD OF THE INVENTION

This application relates generally facial coverings, and in particular to face mask enhancing inserts.

### BACKGROUND

Face masks may be worn to cover, for example, oral and nasal cavities of the wearer to prevent or reduce the transmission of bacteria or viral particles from the wearer's mouth or nose to the surrounding environment, or to prevent or reduce the transmission of such particles from the surrounding environment to the wearer's mouth or nose. Such masks may also be useful in preventing or reducing the transmission of other undesirable particles (e.g., dust, smoke). Face masks are commonly employed in hospital settings to protect healthcare professionals or patients, but may also be used in other contexts, for example, in preventing inhalation or transmission of bacteria or viral particles, or inhalation of smoke or dust, in more routine day-to-day tasks.

We have never before been asked to keep a mask on our faces when out in public or even sometimes in the privacy of our own homes. We have now been asked to wear them for many hours at a time. This is a completely novel concept for a large part of the world. Most people do not enjoy the act of wearing a mask, which is a problem. Given the importance of wearing a mask, we need to be more comfortable while wearing our masks in order to be diligent about keeping them on. Wearing a mask is of upmost importance to keeping people healthy. Additionally, wearing a mask may create a unique environment for our respiratory system. This environment is challenging.

### SUMMARY

The terms “invention,” “the invention,” “this invention” and “the present invention” used in this patent are intended to refer broadly to all of the subject matter of this patent and the patent claims below. Statements containing these terms should be understood not to limit the subject matter described herein or to limit the meaning or scope of the patent claims below. Embodiments of the invention covered by this patent are defined by the claims below, not this summary. This summary is a high-level overview of various embodiments of the invention and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification of this patent, any or all drawings and each claim.

According to certain embodiments of the present inventions, a removable face mask enhancing insert comprises an absorbent material; an adhesive applied to a first side of the absorbent material; and a solution applied to at least a second side of the absorbent material, wherein the solution is configured to disperse from the absorbent material.

In some embodiments, the removable face mask enhancing insert is attachable to a surface of a face mask.

In certain embodiments, the absorbent material is organic.

The absorbent material may comprise wool felt.

The solution, in some embodiments, comprises at least one of aromatherapy scents, vitamins, health supplements, or medications.

The solution, in certain embodiments, is organic.

In some embodiments, the absorbent material is disc-shaped.

The absorbent material may be approximately ⅜ of an inch in diameter.

In certain embodiments, the solution forms an outer coating of at least the second side of the absorbent material.

The dispersing of the solution, in some embodiments, lasts approximately 8 hours.

According to certain embodiments of the present invention, a face mask system comprises a face mask; and a removable face mask enhancing insert comprising an absorbent material; an adhesive applied to a first side of the absorbent material; and a solution applied to at least a second side of the absorbent material, wherein the solution is configured to disperse from the absorbent material.

The removable face mask enhancing insert, in certain embodiments, is attachable to a surface of the face mask.

The surface may comprise an inner surface configured to face a wearer when the face mask is worn by the wearer.

In some embodiments, the removable face mask enhancing insert is interchangeable with a second removable face mask enhancing insert.

In certain embodiments, at least one of the absorbent material or the solution is organic.

The absorbent material may comprise wool felt.

The solution, in some embodiments, comprises at least one of aromatherapy scents, vitamins, health supplements, or medications.

The absorbent material, in certain embodiments, is disc-shaped and approximately ⅜ of an inch in diameter.

The solution may form an outer coating of at least the second side of the absorbent material.

In some embodiments, the dispersing of the solution lasts approximately 8 hours.

### BRIEF DESCRIPTION OF THE DRAWINGS

In the following detailed description, embodiments of the invention are described referring to the following figures:

FIG. 1 illustrates a face mask with an attachable mask insert according to certain embodiments of the present invention.

FIG. 2 illustrates a top view of an attachable mask insert according to certain embodiments of the present invention.

FIG. 3 illustrates a side view of the attachable mask insert of FIG. 2.

FIG. 4 illustrates a face mask with an attachable mask insert worn by a wearer according to certain embodiments of the present invention.

### DETAILED DESCRIPTION

The subject matter of embodiments of the present invention is described here with specificity to meet statutory requirements, but this description is not necessarily intended to limit the scope of the claims. The claimed subject matter may be embodied in other ways, may include different elements or steps, and may be used in conjunction with other existing or future technologies. This description should not be interpreted as implying any particular order or arrangement among or between various steps or elements except when the order of individual steps or arrangement of elements is explicitly described. Directional references such as “up,” “upper,” “lower,” “down,” “top,” “left,” “right,” “bottom,” “among others, are intended to refer to the orientation as illustrated and described in the figure (or figures) to which the components and directions are referencing.

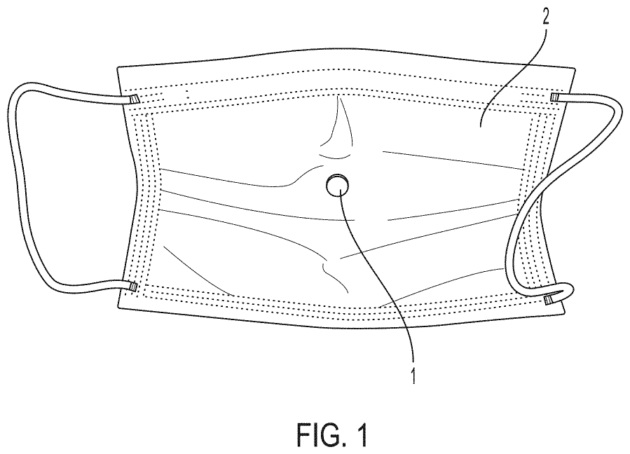

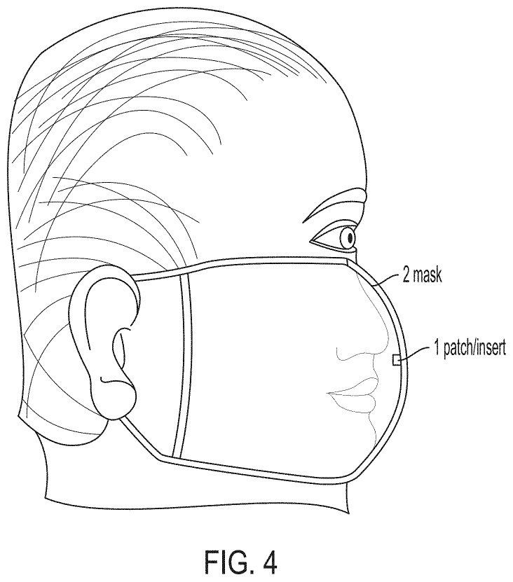

In some embodiments, as shown in FIGS. 1 and 4, a mask enhancing insert 1 may be a small, disc-shaped object that is able to disperse various solutions and to attach to a surface of a face mask 2. The mask enhancing insert 1 not only makes breathing while wearing the face mask 2 more enjoyable but also serves as a vehicle to for dispersing aromatherapy scents, vitamins, health supplements, and medications through inhalation by a wearer. The mask enhancing inserts 1 may provide an opportunity to afford an overall improvement to the wearer's health through the inhalation of the aromatherapy scents, vitamins, health supplements, and medications dispersed by the mask enhancing insert 1.

The attachable mask enhancing insert 1 may be soaked in, coated, sprayed, filled, infused, etc. with a variety of different solutions, e.g., liquid solutions, that include any combination of aromatherapy scents, vitamins, health supplements, medications, etc. The mask enhancing inserts 1 will be a vehicle for an endless array of inhalable purposes. These inhalable purposes may include, but need not be limited to, things as simple as providing a pleasurable scent and aromatherapy in the face mask 2 for the wearer to things as serious as providing inhalable medication or other medicinal purposes. There has never been an opportunity for people to wear a mask enhancing insert 1 in a face mask 2 with inhalable solutions for the benefit of their health and safety. The mask enhancing insert 1 may take advantage of this unique development of mask wearing in our society and make the act of doing so more pleasant and potentially improve the health of the wearer with the mask enhancing inserts 1.

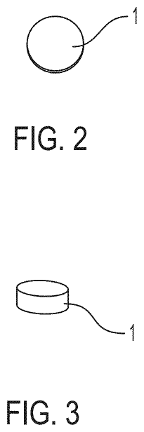

The mask enhancing insert 1, as shown in FIGS. 2 and 3, may vary in shape according to purpose, e.g., the mask enhancing inserts 1 may be circular, ovular, square, rectangular, triangular, etc., and will be approximately ⅜ of an inch in diameter. In some embodiments, the mask enhancing insert 1 may be laminated or coated with an adhesive on one side of the mask enhancing insert 1 to permit the mask enhancing insert 1 to attach or be coupled to the surface of the face mask 2. The surface of the face mask 2 to which the mask enhancing insert 1 faces the wearer when the face mask 2 is worn by the wearer.

Additionally, the side of the mask enhancing insert 1 opposite the adhesive side may have a dissolvable exterior coating, which will dissolve due to the humid environment created within the face mask 2 while a wearer wears the face mask 2 due to the breathing of the wearer. The adhesive is formulated to withstand the humid environment of the face mask 2 when worn, with low VO2. The adhesive is also formulated to be releasable and washable as to not stain a cloth face mask 2 and enable the mask enhancing inserts 1 to be interchangeable depending on the preferences or needs of the wearer.

In some embodiments, the mask enhancing insert 1 may be made out of 100% wool felt, but other suitable materials may be used as well. The wool felt may enable the mask enhancing insert 1 to absorb and hold the solution treatment. The wool felt itself as well as the solution treatment may be 100% natural and organic. For example, a peppermint mask enhancing insert 1 is made from organic wool felt and an organic peppermint solution, e.g., a peppermint essential oil. In some embodiments, there will be one drop of peppermint oil dispensed onto each mask enhancing insert 1.

The mask enhancing insert 1 may be dense enough to hold and absorb enough solution so that the dispersing of the solution while the wearer wears the face mask 2 with the mask enhancing insert 1 attached lasts for approximately 8 hours. The density of the mask enhancing insert 1 also gives it enough body to keep the mask enhancing insert 1 from folding or becoming dislodged inside the face mask 2. The mask enhancing insert 1 may be used for one day and then may be removed and disposed of Removing and disposing of the used mask enhancing insert 1 and replacing it with a new mask enhancing insert 1 daily may avoid germ build up in the face mask 2 and may provide a fresh experience each day.

The mask enhancing inserts 1 be packaged in a resealable bag or other suitable container to maintain freshness.

The mask enhancing insert 1 enable the face mask 2 to remain free of fragrance or solutions which could deteriorate the integrity of the protective quality of the mask, as any solution involved in the inhalation experience is applied directly to the mask enhancing insert 1 instead of in the face mask 2. Applying the solution to the mask enhancing insert 1 instead of directly to the face mask 2 may also protect the face mask 2 from staining by keeping the face mask 2 free of the solution and only the attachable mask enhancing insert 1 would receive the solution, protecting the face mask 2.

The mask enhancing insert 1 may be used on disposable face masks 2 or face masks 2 that are reusable.

The mask enhancing insert 1 may become a revolutionary tool for delivering inhalable solutions with a broad spectrum of applications. For example, the mask enhancing insert 1 may include flavors geared towards children like birthday cake, cinnamon bun, tropical fruit, sweet and sour, etc.

The mask enhancing insert 1 may include flavors for better breathing, such as eucalyptus, spearmint, lemon mint, etc.

The mask enhancing insert 1 may include flavors for aromatherapy, such as lavender, rosemary, sandalwood, citrus, etc.

The mask enhancing insert 1 may include flavors to correlate with the seasons, such as pumpkin pie, candy cane, eggnog, cotton candy, piña colada, key lime pie, etc.

The mask enhancing insert 1 may include flavors for energy, such as coffee, espresso, latte, etc.

In certain embodiments, the mask enhancing insert 1 may be provided without any solution and instead may be provided with a variety of flavors in a small vial for consumers to apply based on their choice of flavors for the day. This may allow the consumer to have the flexibility to use different scents. If the consumer is having an anxiety ridden day, the consumer may use lavender to scent the mask enhancing insert 1. If the consumer is having a day where they need extra energy, the consumer may use an energy flavor to scent the mask enhancing insert 1. If the consumer is having a day where they need extra vitamin C, the consumer may use a vitamin infused flavor for mask enhancing insert 1. If the consumer would like to use a sentimental flavor for the holidays, the consumer may use a flavor for the holidays to scent the mask enhancing insert 1. If the consumer needs to inhale a medication, the consumer may use a medication infused mask enhancing insert 1.

It is to be understood that the above description is intended to be illustrative and not restrictive. Many embodiments will be apparent to those of skill in the art upon reviewing the above description. The scope of the invention should, therefore, be determined not with reference to the above description, but instead should be determined with reference to the appended claims along with their full scope of equivalents.

While the foregoing disclosure shows illustrative aspects of the disclosure, it should be noted that various changes and modifications could be made herein without departing from the scope of the disclosure as defined by the appended claims. The functions, steps and/or actions of the method claims in accordance with the aspects of the disclosure described herein need not be performed in any particular order. Furthermore, although elements of the disclosure may be described or claimed in the singular, the plural is contemplated unless limitation to the singular is explicitly stated.

1. A removable face mask enhancing insert comprising:
an absorbent material;
an adhesive applied to a first side of the absorbent material; and
a solution applied to at least a second side of the absorbent material, wherein the solution is configured to disperse from the absorbent material.

2. The removable face mask enhancing insert of claim 1, wherein the removable face mask enhancing insert is attachable to a surface of a face mask.

3. The removable face mask enhancing insert of claim 1, wherein the absorbent material is organic.

4. The removable face mask enhancing insert of claim 3, wherein the absorbent material comprises wool felt.

5. The removable face mask enhancing insert of claim 1, wherein the solution comprises at least one of aromatherapy scents, vitamins, health supplements, or medications.

6. The removable face mask enhancing insert of claim 1, wherein the solution is organic.

7. The removable face mask enhancing insert of claim 1, wherein the absorbent material is disc-shaped.

8. The removable face mask enhancing insert of claim 7, wherein the absorbent material is approximately ⅜ of an inch in diameter.

9. The removable face mask enhancing insert of claim 1, wherein the solution forms an outer coating of at least the second side of the absorbent material.

10. The removable face mask enhancing insert of claim 1, wherein the dispersing of the solution lasts approximately 8 hours.

11. A face mask system comprising:
a face mask; and
a removable face mask enhancing insert comprising:
an absorbent material;
an adhesive applied to a first side of the absorbent material; and
a solution applied to at least a second side of the absorbent material, wherein the solution is configured to disperse from the absorbent material.

12. The face mask system of claim 11, wherein the removable face mask enhancing insert is attachable to a surface of the face mask.

13. The face mask system of claim 12, wherein the surface comprises an inner surface configured to face a wearer when the face mask is worn by the wearer.

14. The face mask system of claim 11, wherein the removable face mask enhancing insert is interchangeable with a second removable face mask enhancing insert.

15. The face mask system of claim 11, wherein at least one of the absorbent material or the solution is organic.

16. The face mask system of claim 15, wherein the absorbent material comprises wool felt.

17. The face mask system of claim 11, wherein the solution comprises at least one of aromatherapy scents, vitamins, health supplements, or medications.

18. The face mask system of claim 11, wherein the absorbent material is disc-shaped and approximately ⅜ of an inch in diameter.

19. The face mask system of claim 11, wherein the solution forms an outer coating of at least the second side of the absorbent material.

20. The face mask system of claim 11, wherein the dispersing of the solution lasts approximately 8 hours.
